(19) United States
(12) Patent Application Publication
(10) Pub. No.: US 2014/0379610 A1
(43) Pub. Date: Dec. 25, 2014

(54) Cannabis Product Updated User Feedback System and Method

(71) Applicant: Thomas K. Lindeman, Bellevue, WA (US)

(72) Inventor: Thomas K. Lindeman, Bellevue, WA (US)

(21) Appl. No.: 14/309,741

(22) Filed: Jun. 19, 2014

Related U.S. Application Data
(60) Provisional application No. 61/837,157, filed on Jun. 19, 2013.

(51) Int. Cl. G06Q 30/02; A61K 36/185

(52) U.S. Cl. CPC G06Q 30/0282; A61K 36/185
USPC 705/347

(57) ABSTRACT

A cannabis product updated user feedback system and method that informs users of the anticipated psychological and physiological effects of a cannabis product. The system includes the creation of an effects graph associated with the cannabis product that may be distributed with the cannabis product or presented on a website. The effects graph includes one or more line graphs depicting the effects and interactions experienced by all reporting users of the product. The system includes a website in which the feedback information regarding a particular cannabis product is collected. The administrator of the website then reviews the feedback information and provides updated line graph data. Each line graph shows the intensity of user-reported effects graded from 0 to an upper limit on the y axis over a time presented on the x axis. The effects graph may include icons that visually portray what a user consumed and how they felt. By viewing the line graph, a user can anticipated the effects, the intensity, when they occur and the duration.

This utility patent application is based on and claims the filing date benefit of U.S. provisional patent application 61/837157 filed on Jun. 19, 2013.

Notice is given that the following patent document contains original material subject to copyright protection. The copyright owner has no objection to the facsimile or digital download reproduction of all or part of the patent document, but otherwise reserves all copyrights.

### BACKGROUND OF THE INVENTION

1. Field of the Invention

This invention pertains to medicinal and therapeutic products that contain one or more active ingredients from the cannabis plant distributed to users, and more particularly to visual review and tracking systems and methods for cannabis products that inform users of the anticipated physiological, psychological effects produced by the product when used in the recommended manner.

2. Description of the Related Art

Many therapeutic and medicinal plant based remedies, such as those from the cannabis plant, are complex and contain several active ingredients. The cannabis plant, also known as cannabis or marijuana, is a complex plant which contains over 50 psychotropic chemicals, known as cannabinoids in varying ratios. The most well-known cannabinoid is delta-9 tetrahydro-cannabinol, also known as THC.

They are many ways to administer therapeutic and medicinal active ingredients including, inhalation, injection, topical application, and oral ingestion. The physiological and psychological effects experienced by users who have consumed products containing active ingredients are very subjective. The subjective effects are directly related to user tolerance, and quantity consumed. They may also be affected by interactions with other drugs or medicines, activities, moods, stress, anxiety, age, sex, race, eating habits, etc., over a period of time. The onset, intensity or duration of the product effects and especially the negative effects associated with the active ingredients, such as increased heart rate, hallucinations, and anxiety, also vary greatly and can be severely affected by consumption of other products or medicines which also contain active ingredients. The user's current mood, activities, eating habits, and environmental factors can also impact the user's experiences in a positive or negative way.

Because the physiological and psychological effects can vary, knowing the anticipated intensity of the product effects, its characteristics, onset and duration, interactions with the above stated conditions and what other users of the product have experienced over a period time would be desirable to a new user. It would also be desirable if this information was readily available to the user via a web page, a product label, a marketing brochure etc. Easy to view and understand depictions of active ingredient effects and interactions for specific cannabis products are often not readily available today.

Besides the wide variety of powerful pharmaceutical medicines on the market and over-the-counter drugs such as cold remedies, diet products, pain relievers, and energy products, multiple states have now legalized cannabis products for medical use and recreational adult use. Since the user experience of medicinal or therapeutic products containing active ingredients is largely subjective and cannot be accurately predicted using measurements and data such as grams of active ingredient per portion, these factors alone are not sufficient for sellers of products containing active ingredients to adequately inform their customers about the effects. Therefore, an easy to understand visualrepresentation of the user experiences and testimonial information from a population of reporting users of the active ingredients of a particular product and vintage and possible adverse interactions with other products is beneficial consumer safety information.

What is needed is a product information and review system for informing users of therapeutic or medicinal cannabis products with active ingredients of the anticipated positive and negative psychoactive and physiological effects of that product over a period of time. What is also needed is a system that enables a population of users of a product containing active ingredients to easily report positive and negative reactions and interactions with the product for others to review which can then be tabulated, averaged and published on labels or brochures distributed with the product, or on a web site or other digital media including social media such as Twitter or Facebook.

### SUMMARY OF THE INVENTION

At the heart of the invention is the discovery that the psychological, physiological and interaction effects of natural or synthetic cannabis products containing active ingredients varies, and that merely providing objective measurement data, such as the overall amount or percentage of the active ingredients, does not adequately inform users of the overall possible positive or negative effects of the product. Also, merely providing information regarding possible interactions the active ingredients may have with other drugs, caffeine, alcohol, tobacco, or that they may cause drowsiness or gastric-intestinal symptoms is insufficient. Instead, what is needed is quantitative subjective experience data provided for cannabis products which in combination with objective data such as High Pressure Liquid Chromatography (HPLC) technology provides users with a more complete picture of the expected effects Implementation reference example:

The system includes the manufacturing and distribution of a medicinal or therapeutic product that contains active ingredients that have psychological and physiological effects. The product is distributed in an outer container with an effects graph printed on the product packaging or on a product label attached to the outer container or presented on a marketing brochure or product website. The effects graph may include the name of the product, and the types and amount of active ingredients or other product information designed to educate and inform the user.

The effects graph includes at least one line graph with x and y axis depicting one or more subjective psychological and physiological effects of the active ingredients found in the product. By reviewing the line graph before consuming the product, the user may anticipate the timing and onset, intensity, and duration of the psychological, physiologic effects of the active ingredients.

In one embodiment, the line graphs are color coded with the intensity of user effects graded from 0 to an upper limit on the y axis over a time presented on the x axis and optional icons that visually portray consumed products and the subjective experience. By viewing the line graph, a consumer unfamiliar with the product can determine the anticipated psychological and physiological effects, the strength or intensity of these effects, and their duration of action. This information is key safety feature and is particularly needed for cannabis products which is more widely consumed by the population.

The line graph displays averaged information collected from a population of users who report the effects of the product and its interactions with other drugs, activities, moods, eating habits, etc., over a period of time. The line graph may be displayed directly on the product package, product label, or marketing material attached to the product or presented on other media such as a website. The product package or website will also include a uniquely generated QR code that links back to the specific product entry in an online catalog where users can write product reviews, create their own experience curves, and read more safety and usage information about the product.

The information for the line graph may be produced by subjective clinical trials administered by the product manufacturer or distributor. Alternatively, disclosed is an alternative method for generating the data by allowing experienced users of a product to report their experiences to a website operated by a third party, or product owner, seller or distributor. In one embodiment, experience users can visit the specific website for a product and submit feedback information regarding the product. The website is linked to a database engine that can produce a personal line graph based on the single experience user's inputted information. The information from the experience user may also be combined with data from a plurality experience users of the produce to produce a line graph from a plurality of experience users. Both line graphs can then be saved in a personal or general file on the database. When a large sampling of line graphs for specific products containing active ingredients has been saved in the database, the product owner or distributor may compiled average line graph that can be distributed with the product or on a website or other media.

In the preferred embodiment, the above described system and method is used with cannabis containing products that containing cannabis from different plants, different growing regions, and cultivated using different methods.

### DESCRIPTION OF THE DRAWINGS

FIG. 1 is an illustration of the cannabis product updated user feedback system showing a physical product containing at least one cannabis ingredient, a brochure for the product, and a website coupled to a database.

FIG. 2 is an illustration of an effects label.

FIG. 3 is an illustration of a user using a mobile computing device or phone with a touch screen to input physiological and psychological effect information into plank line graph template then uploaded via a wide area network to database container folders of specific cannabis products.

FIG. 4 is an illustration of an effects graph uploaded by a user showing one line graph and a plurality of icons and emoticons that provide more information about what other products with active ingredients, e.g. alcohol, was consumed and when, what the intensity and feeling of the experience was, and what activities the user undertook that could affect the overall experience.

### DESCRIPTION OF THE EMBODIMENT(S)

A system and method for informing users of the anticipated psychological and physiological effects of a medicinal or therapeutic product containing active chemical ingredients by distributing with product materials or providing via a website an effects graph with at least one line graph depicting the psychological and physiologic effects or experiences. The data used to produce the line graph may be collected by a 3^{rd} party or product manufacturer or distributor of from reports uploaded to a website by other users after the specific active ingredients have been consumed.

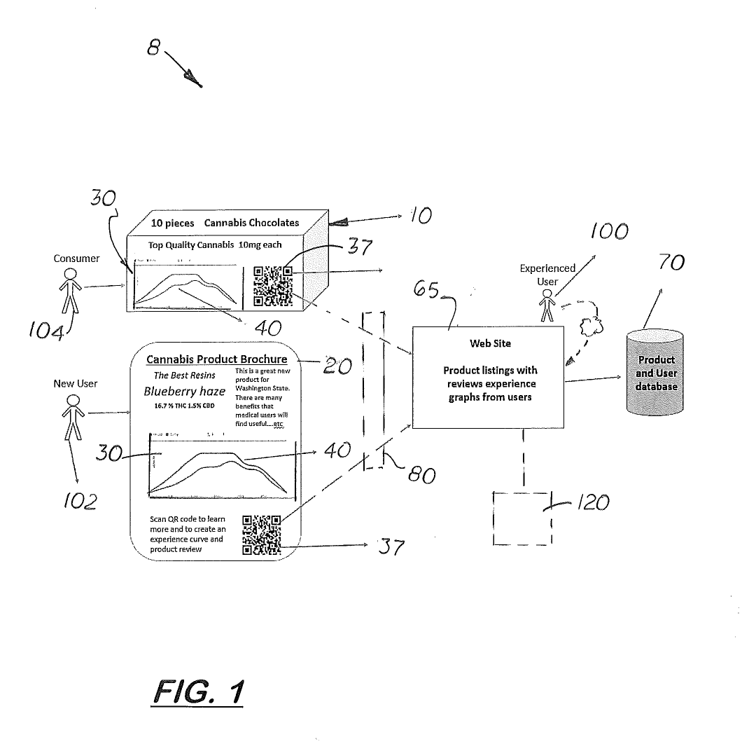

The system 8 and method may be used with various types of active ingredients and products containing one or more active ingredients. FIG. 1 is an illustration of the cannabis product updated user feedback system 8 showing a physical product 10 containing at least one cannabis ingredient, a brochure for the product, and a website coupled to a database.

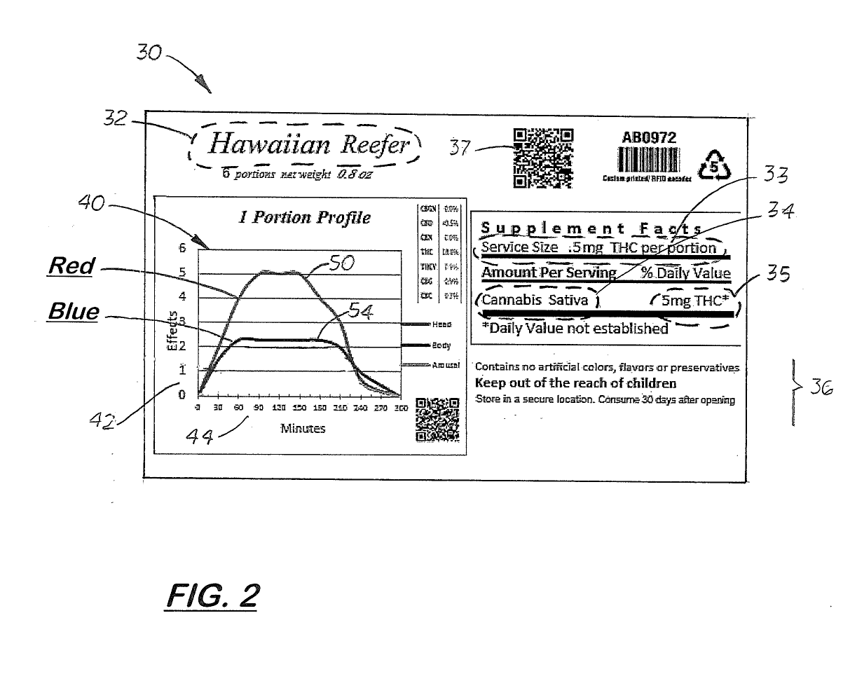

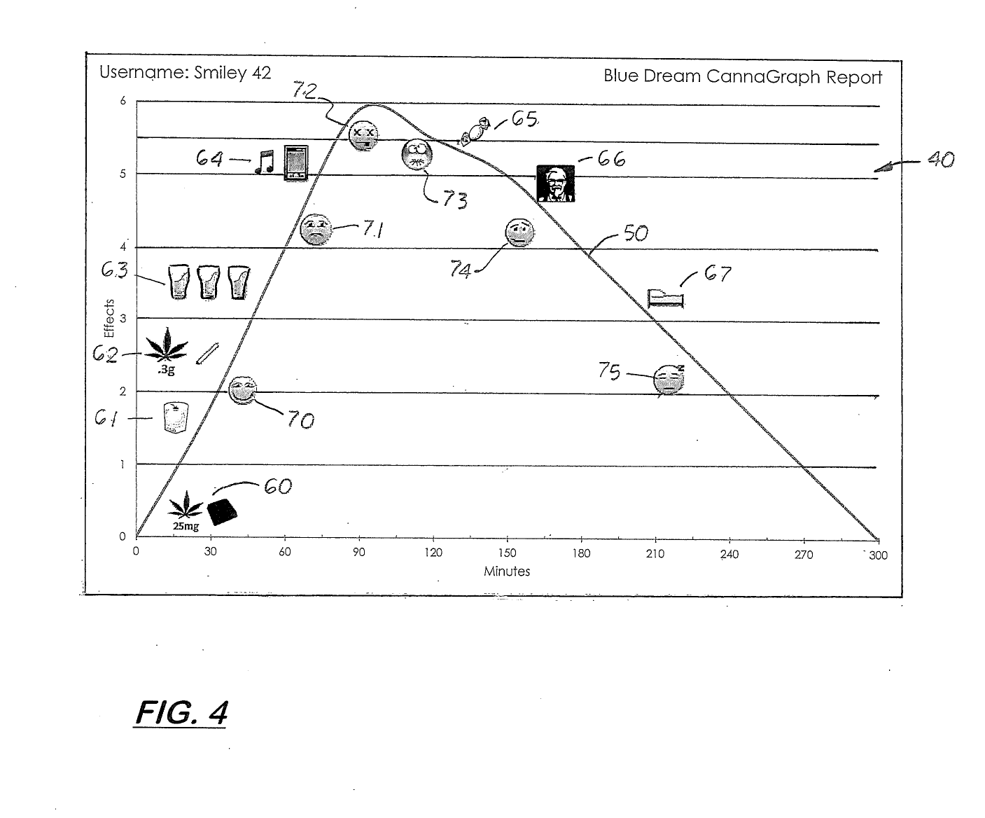

The system 8 includes the creation of an effects graph 40 associated with the cannabis containing product 10 that may be distributed with the cannabis product 10, a brochure 20, or presented on a website 65. The effects graph 40 includes one or more line graphs 50 (see FIGS. 2 and 4) depicting the effects and interactions experienced by all reporting users of the product 10. The system 8 includes a website 65 in which the feedback information regarding a particular cannabis product 10 is collected and saved in database 70. Associated with the website 65 is a specific product webpage 120 on which specific effects graphs associated with the product 10 are shown along with other pertinent information. The administrator of the website 65 then reviews the feedback information and provides updated line graph 50. Each line graph 50 shows the intensity of user-reported effects graded from 0 to an upper limit on the y axis over a time presented on the x axis. The effects graph 40 may include icons 61-75 that visually portray what a user consumed and how they felt. By viewing the line graph, a new user 102 or a information seeking consumer 104 can anticipated the effects, the intensity, when they occur and the duration.

As more clearly shown in FIG. 2, the printed label 30 includes information required by state and federal regulations, such as the common name of the cannabis product 32, the serving size and the amount of THC per serving 33, the total amount of THC in the container 34, and the consumption or warnings 35. In some instances, a batch or lot number or other product information may be provided. To be in compliance with new state laws in WA and CO, orally consumable products must also include the ingredients, possible allergens, the serving size, and other nutritional information or safety warnings.

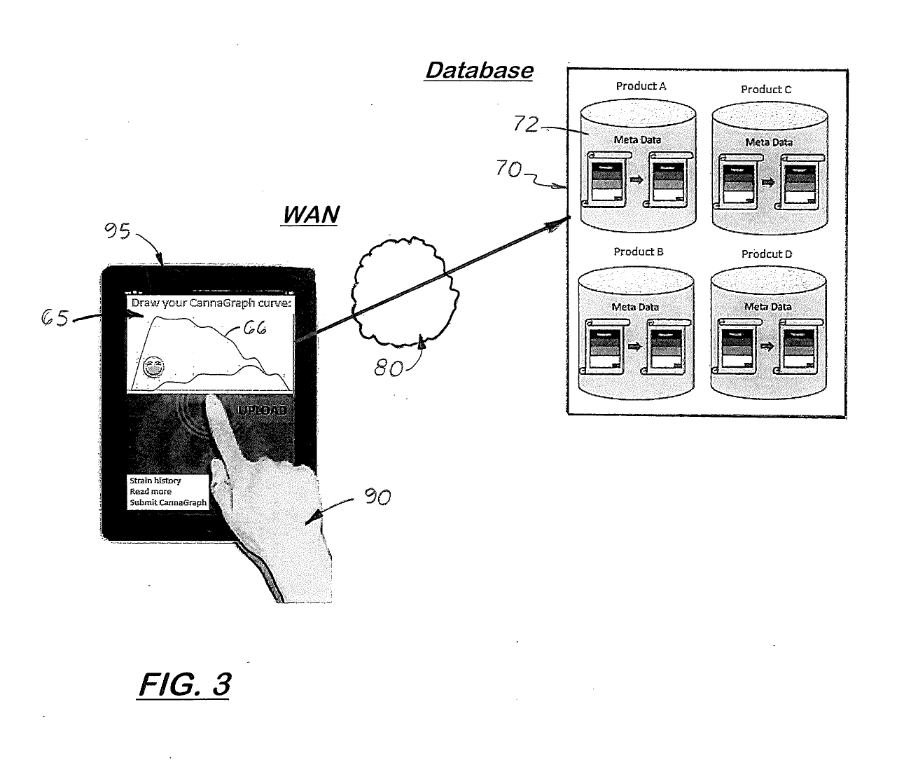

As shown in FIG. 2, the printed label 30 also includes an effects graph 40 that depicts one or more line graph lines (three line graphs are shown denoted by reference numbers 50, 54, 58). The line graph lines 50, 54 are color coded (BLUE and GREEN, respectively) each associated with one of the following: the psychological (head) effect; physiological (body) effect. The upper lint line 58 (color coded RED) is a danger zone line which shows that the user will be very uncomfortable or sick if their experience reaches this level. The effects graph 40 includes a graded intensity effect using a scale from 0 to 5 on the y axis 42 over time (in minutes) indicated along the x axis 44. By viewing the line graphs 50,54, and 58 on the effects graph 40, a user can quickly determine prior to use, the anticipated the above listed effects of when, how and the duration after consuming or smoking the cannabis product. The effects graph 40 may be printed directly on the printed label 30 attached to the container 20, or to the product's outer wrapper. In the preferred embodiment, the label 30 may also include a barcode or QR code 37 that when scanned, transfers the user to the website 65 associated with the product. The effects graph 40 may also be presented on a website 65 (see FIG. 3) linked to the product.

Initially, the information used to produce the line graphs 50 and 54a are collected by the product manufacturer or distributor. As usage of the product 10 increases, reports may be collected from a population of users then used to update the data using the average data for that population. FIG. 3 is an illustration of a user using a touch screen enabled mobile computing device 95 to input physiological and psychological effect information into a line graphic template 66 presented on a website 65 that is then uploaded via internet connection 80 to a database 70 container folders 72 of other products.

The uploaded graphic templates 66 are collected and the collected line graph lines 50 and 54, can be continuously updated and revised if necessary, so the effects graph 40 distributed with the cannabis product is accurate and represents a crowd sourced average.

In order to submit a report or review other reports, the experience user 100, new user 102 or consumer 104 visits a 3^{rd} party, or product manufacturer's or distributor's website 65. If the experience user 100 wants to input information, the experience user 100 may be setup a user reporting account. Once at the website 65, the user 100, 102, or 104 can navigate to the specific product reporting page 1210 that contains more information about the product 10 including cannabis strain information, test results, etc and other experience customer reports. If a QR code or bar code 37 is used on the printed label 30, the user may use a scanning device to connect directly to the specific product page 120. In one embodiment, a 3^{rd} party, or the product manufacturer or distributor creates a database 70 for tracking and updating all information about that product 10, including all user uploaded reports, text entries, ratings, comments, etc. The 3^{rd} party, product manufacturer or distributor then monitors the uploaded reports regularly. The QR code or bar code 37 may include an encrypted security token which tell the web site to enable to functionality for allowing the user to create their own review and experience curves. Website links can also include this security access token.

As stated above, the user's consumption of other active ingredients, activities, eating and drinking habits, or moods and environmental factors can also impact the user's experience. This is especially problematic with cannabis containing products. FIG. 4 depicts an effects graph 40 for a cannabis product 10 that shows a reported line graph 50 from an experience user 100 showing the graded psychological or physiologic effect over time, and a plurality of icons 61-67 indicating the user's consumption of alcohol and food, and physical activities and emoticons that indicate the user's moods, or mental states, consumption or sleeping activities.

In compliance with the statute, the invention described has been described in language more or less specific as to structural features. It should be understood however, that the invention is not limited to the specific features shown, since the means and construction shown, comprises the preferred embodiments for putting the invention into effect. The invention is therefore claimed in its forms or modifications within the legitimate and valid scope of the amended claims, appropriately interpreted under the doctrine of equivalents.

I claim:

1. A cannabis product updated user feedback system, comprising:
a. a product containing at least one active cannabis ingredient;
b. an effects graph associated with the cannabis product, the effects graph including at least one line graph showing the intensity over time of one of the following: the psychological effect, the physiological effect, or the subjective experiences.
c. a website configured to receive feedback information from visitors regarding the intensity of the cannabis ingredient over time of one of the following: the psychological effect, the physiological effect, or the subjective experiences, said feedback information then used to create at least one line graph on the effects graph.

2. The system as recited in claim 1, wherein the effects graph is displayed the product, a label attached to the product, a container for the product, a brochure associated with the product, or on a webpage associated with the product or the cannabis ingredient.

3. The system as recited in claim 1, wherein said effects graph is presented on a website or digital media associated with said product.

4. A method of presenting updated information regarding the psychological and physiological effects of a product containing one or more active cannabis ingredients, comprising the following steps:
a. providing a website listing a plurality of products containing at least one cannabis ingredient, each said product or cannabis ingredient being associated with an effects graph;
b. a database coupled to the website containing a plurality of data storage directories for each specific product containing at least one cannabis ingredient, each data storage directory containing feedback information from users of the specific product regarding the intensity of the psychological effect, the physiological effect, or the subjective experiences of a user over time, the feedback information from all users then combined and used to create an updated effects graph with at least one line graph associated with the cannabis ingredient depicting the intensity of the psychological effect, the physiological effect, or the subjective experiences of a plurality of users over time; and,
c. presenting the updated effects graph on the website or on the product, or on a label attached the product or an outer container for the product, or on printed substrate associated with the product.

5. The method as recited in claim 4, wherein said website includes an experience user input webpage where experience users of the product input information regarding the intensity of the psychological effect, the physiological effect, or the subjective experiences over time
